(19) United States
(12) Patent Application Publication
(10) Pub. No.: US 2023/0146391 A1
(43) Pub. Date: May 11, 2023

(54) AIRBORNE DELIVERY OF MICROROBOTS

(71) Applicant: Colorado School of Mines, Golden, CO (US)

(72) Inventors: David Marr, Golden, CO (US); Keith Neeves, Golden, CO (US)

(21) Appl. No.: 17/983,933

(22) Filed: Nov. 9, 2022

Related U.S. Application Data
(60) Provisional application No. 63/277,598, filed on Nov. 9, 2021.

(51) Int. Cl. A61M 11/00

(52) U.S. Cl. CPC A61M 11/003; A61M 11/001

(57) ABSTRACT

Methods and systems are provided for aerosolization of individual building blocks of medical microrobots and subsequent in situ assembly into microrobots capable of medical intervention deep within lung tissues of a subject. The methods and systems of the disclosure may allow for microrobot-based therapy of pulmonary diseases that have previously been difficult to effectively treat using conventional therapeutic approaches.

### CROSS REFERENCE TO RELATED APPLICATIONS

This application claims priority to U.S. Provisional Patent Application 63/277,598, filed 9 Nov. 2021, the entirety of which is hereby incorporated by reference.

### STATEMENT REGARDING FEDERALLY SPONSORED RESEARCH

This invention was made with government support under grant numbers R21AI138214 and RO1NS102465 awarded by the National Institutes of Health. The government has certain rights in the invention.

### FIELD

This disclosure relates generally to microrobots capable of medical intervention, and particularly to methods and systems for airborne delivery of microrobots by aerosolizing individual building blocks of the microrobots and assembling the microrobots in situ.

### BACKGROUND

The promise of micro-scale robotic devices capable of medical intervention has led to the development of medical microrobots (“microbots” or “μbots”) that can swim, crawl, and roll. Generally ranging in size from the tens to the thousands of micrometers and designed for movement and delivery through the blood stream or gastrointestinal tract, microbots have potential applications ranging from disease diagnosis to targeted therapies for stroke and cancer.

For lung diseases in particular, delivery through the airway via an aerosol provides the most direct route of administration, both for microbots and for conventional therapeutics. Aerosol-based therapies have been used for centuries to treat diseases such as asthma, angina, and persistent cough, and with the advent of metered-dose inhalers beginning in the 1950s, adoption of these therapies has significantly increased. However, the efficiency and effectiveness of aerosolized treatment is significantly reduced in diseases where fluid buildup creates transport barriers to underlying biofilms and epithelial cells, such as pneumonia, cystic fibrosis, acute bronchitis, and chronic obstructive pulmonary disorder. Given their potential to enhance in vivo transport, microbots could be used to overcome these impediments and enhance treatments. Most commonly, microbots are controlled via magnetic fields that are not strongly attenuated by tissues, and microbots that are capable of direct translation by swimming and/or rolling to deliver drugs in an aqueous environment have been developed.

Delivery through air for lung-based therapies requires certain considerations that limit the use of most current microbot delivery strategies. Primary among these considerations, and in general the key to effective inhalation-based therapy, is particle size—particles or droplets that are too large cannot remain suspended in air long enough to reach deep within the lungs, while particles or droplets that are too small are not effectively held in the lungs and are simply exhaled. The optimum aerodynamic particle size for drug-laden aerosols is generally between about 1 μm and about 5 μm, and particles are commonly delivered via nebulizer to define a desired particle size distribution to control the deposition profile within the lungs. At these length scales, however, viscosity plays a dominant role in locomotion; microorganisms overcome this using physical adaptations, such as rotating flagella, that are difficult to artificially replicate and control. Thus, microbots in the ideal size range are challenging to fabricate and have, to this point, lacked the speed and power necessary to perform work throughout the pulmonary network of a subject.

### SUMMARY

In an aspect of the present disclosure, a method for delivering microrobots comprises (a) providing a liquid suspension comprising superparamagnetic particles of at least one microrobot building block; (b) aerosolizing the liquid suspension to form aerosolized droplets; (c) administering the aerosolized droplets into a target volume; and (d) applying a first magnetic field to the aerosolized droplets to cause the superparamagnetic particles to aggregate into microrobots in the form of microwheels.

In embodiments, the target volume may be within a body of an animal subject and the microrobots may be medical microrobots capable of performing a medical intervention within the target volume. The animal subject may, but need not, be a human. The target volume may, but need not, comprise a pathway or space within a pulmonary or respiratory system of the animal subject, and may, but need not, comprise at least one of lower bronchioles of the animal subject and alveolar spaces of the animal subject. The medical intervention may, but need not, comprise delivering an active pharmaceutical ingredient to the target volume.

In embodiments, the method may further comprise directing the microwheels to a preselected sub-volume of the target volume by applying a second magnetic field.

In embodiments, the liquid suspension may be aerosolized, and the aerosolized droplets may be administered, using a nebulizer.

In embodiments, a mean or median particle size of the superparamagnetic particles may be between about 1 μm and about 5 μm.

In another aspect of the present disclosure, a method for performing a medical intervention within a body of an animal subject comprises (a) providing a liquid suspension comprising superparamagnetic particles of at least one microrobot building block; (b) aerosolizing the liquid suspension to form aerosolized droplets; (c) administering the aerosolized droplets into a target volume within the body of the subject; and (d) applying a first magnetic field to the aerosolized droplets to cause the superparamagnetic particles within the target volume to aggregate into microrobots in the form of microwheels, wherein the microrobots are medical microrobots capable of performing a medical intervention within the target volume.

In embodiments, the animal subject may be a human.

In embodiments, the target volume may comprise a pathway or space within a pulmonary or respiratory system of the animal subject. The target volume may, but need not, comprise at least one of lower bronchioles of the animal subject and alveolar spaces of the animal subject.

In embodiments, the medical intervention may comprise delivering an active pharmaceutical ingredient to the target volume.

In embodiments, the method may further comprise directing the microwheels to a preselected sub-volume of the target volume by applying a second magnetic field.

In embodiments, the liquid suspension may be aerosolized, and the aerosolized droplets may be administered, using a nebulizer.

In embodiments, a mean or median particle size of the superparamagnetic particles may be between about 1 μm and about 5 μm.

In another aspect of the present disclosure, a system for delivering microrobots comprises a liquid suspension comprising superparamagnetic particles of at least one microrobot building block; a nebulizer, configured to aerosolize the liquid suspension to form aerosolized droplets and administer the aerosolized droplets into a target volume; and a magnetic actuation device, configured to apply a first magnetic field to the aerosolized droplets to cause the superparamagnetic particles within the target volume to aggregate into microrobots in the form of microwheels.

In embodiments, the target volume may be within a body of an animal subject and the microrobots may be medical microrobots capable of performing a medical intervention within the target volume. The medical intervention may, but need not, comprise delivering an active pharmaceutical ingredient to the target volume.

In embodiments, the magnetic actuation device may be further configured to direct the microwheels to a preselected sub-volume of the target volume by applying a second magnetic field.

In embodiments, a mean or median particle size of the superparamagnetic particles is between about 1 μm and about 5 μm.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to about 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIG. 1A is an illustration of microwheel rotation and translation over a period of 2 seconds under an applied alternating current (AC) magnetic field.

FIG. 1B is a graph showing the dependence of microwheel velocity (upper data points and line) and power (lower data points and line) on assembled microwheel size.

FIG. 1C is a graph showing the dependence of microwheel rotation rate on the inverse of microwheel radius.

FIG. 2A is a concept illustration of a microwheel assembly strategy according to embodiments of the present disclosure.

FIG. 2B is a measured size distribution of aerosolized droplets, with droplets containing beads of microbot building blocks identified.

FIG. 2C is a graph showing pre- and post-aerosolization microwheel sizes and velocities.

FIG. 2D is an optical microscopic image of a sample of a microbot building block aerosol in oil.

FIG. 2E is a histogram of microwheel velocities.

FIG. 3A is an illustration of aerosolization of microbot building block bead-containing droplets into a 3D-printed lung model.

FIG. 3B is a false-color image of fluorescent, superparamagnetic beads dispersed throughout the lung model of FIG. 3A after aerosolization.

FIG. 3C is an illustration of the lung model of FIG. 3A and the beads of FIG. 3B upon application of a rotating magnetic field.

FIG. 3D is an illustration of the beads of FIG. 3B traveling down pathways of the model lung of FIG. 3A.

FIG. 3E is an illustration of the beads of FIG. 3B captured by a permanent magnet to form a bolus within the lung model of FIG. 3A.

FIG. 3F is an illustration of the lung model of FIG. 3A and the bolus of FIG. 3E upon removal of the permanent magnet and subsequent application of a weak AC magnetic field.

FIGS. 3G and 3H are illustrations of the bolus of FIG. 3E targeted to specific branches or pathways within the model lung of FIG. 3A by application of the weak AC magnetic field.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

As used herein, unless otherwise specified, the term “colloid” refers to a mixture in which particles of one substance (the “dispersed phase”) are dispersed throughout a volume of a different substance (the “dispersion medium”). Where the dispersed phase and the dispersion medium of a colloid are specifically identified herein, they are separated by a hyphen, with the dispersed phase identified first, e.g. a reference herein to a “water-air colloid” refers to a colloid in which water is the dispersed phase and air is the dispersion medium.

As used herein, unless otherwise specified, the term “aerosol” refers to a colloid in which the dispersed phase is a liquid and the dispersion medium is a gas. Examples of aerosols as that term is used herein include but are not limited to clouds, condensation, fog, hair spray, and mist.

As used herein, unless otherwise specified, the term “one-dimensional” refers to an object or structure having two spatial dimensions that are minimal or negligible when compared to a third spatial dimension of the same object or structure. Thus, by way of non-limiting example, a “one-dimensional” object or structure may have a length and a width that are minimal or negligible in comparison to its height, or a height and a width that are minimal or negligible in comparison to its length, or a height and a length that are minimal or negligible in comparison to its width.

As used herein, the term “pharmaceutical composition” refers to a composition of matter comprising at least one active pharmaceutical ingredient that is adapted or configured to be administered to an animal for a therapeutic purpose.

As used herein, the term “subject” refers generally to an animal, including but not limited to a human, to which a composition or formulation provided by the present disclosure is administered or is to be administered. Other animals that may be “subjects” as those terms are used herein include but are not limited to companion animals, such as cats, dogs, and horses; livestock animals, such as cattle, goats, sheep, and pigs; mice; and rats.

As used herein, unless otherwise specified, the term “two-dimensional” refers to an object or structure having one spatial dimension that is minimal or negligible when compared to either or both of two other spatial dimensions of the same object or structure. Thus, by way of non-limiting example, a “two-dimensional” object or structure may have a height that is minimal or negligible in comparison to its length and/or width, or a length that is minimal or negligible in comparison to its height and/or width, or a width that is minimal or negligible in comparison to its height and/or length.

The present disclosure provides methods and systems that uncouple the size of microbots from their structure and function, and thereby overcome the drawbacks of prior microbot delivery methods and systems, by aerosolizing individual building blocks of microbots and subsequently assembling these building blocks in situ into microbots capable of translation and mechanical work deep within the lung tissues of a subject. The present disclosure thus enables microbot-based therapies for a variety of pulmonary diseases that have previously been difficult to treat.

More particularly, the methods and systems of the present disclosure utilize a non-biomimetic approach, in which microbots are rapidly and reversibly fabricated and powered by assembling micrometer-scale superparamagnetic beads into microbots in the form of microscale wheels (“microwheels” or “μwheels”) upon application of a rotating alternating-current (AC) magnetic field; the beads can be provided with a variety of surface functional groups, which allows for attachment of a variety of pharmaceutical agents to the surface of the microwheels to utilize the microwheels as a drug delivery vehicle. These microwheels roll rapidly and can be immediately redirected with a simple alteration in the orientation of the magnetic field to cause a change in the microwheels' speed and heading. Because the methods and systems of the present disclosure rely on the assembly and rotation of microbots in a weak magnetic field, they do not require the high fields or strong field gradients necessary for magnetophoresis, and as a result the infrastructure required to practice the methods and systems is greatly reduced.

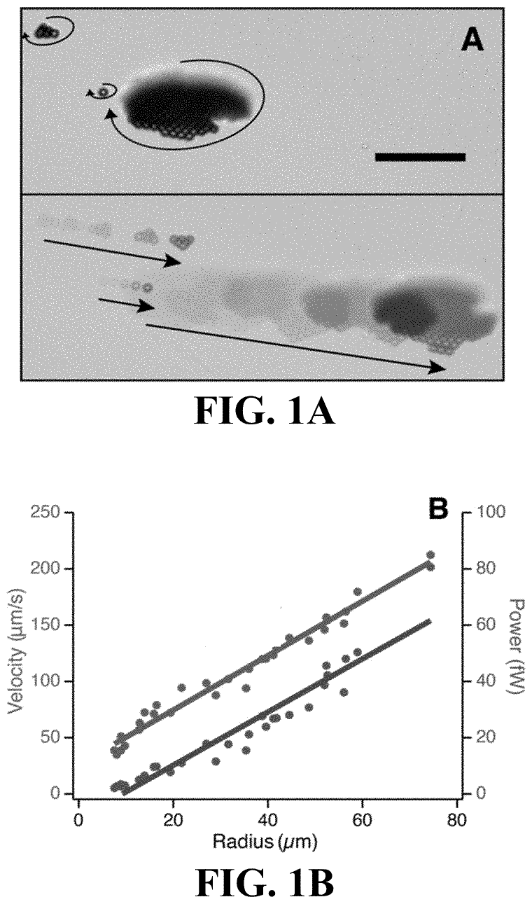

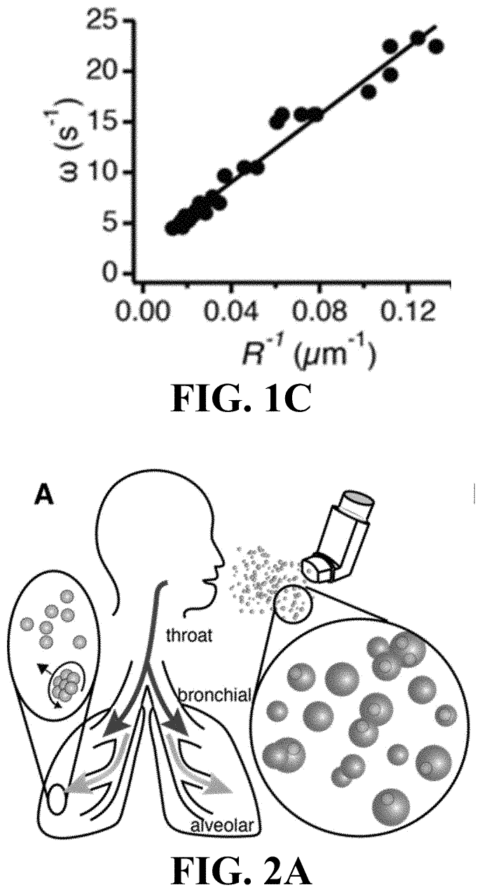

In embodiments of the methods and systems of the present disclosure, upon application of a magnetic field, superparamagnetic beads are brought together via strong attractive interactions to assemble the beads into two-dimensional microwheel structures having varying shapes and sizes. With rotation of the magnetic field, these microwheel structures spin and, with orientation of the field axis off the surface normal, translate, as illustrated in FIG. 1A. Under fixed applied field conditions, the radius R, the rotation rate ω, and the translational velocity V together determine the power P via the rotational torque required to cause the microwheel to spin. With microwheels powered by rotating magnetic fields of magnitude H, the magnetic torque induced can be expressed as τ=Nνμₒχ″H², where N is the number of beads in the microwheel, ν is the volume of an individual bead, to is the permittivity of free space, and χ″ is the imaginary part of the magnetic susceptibility. By approximating the viscous rotational microwheel torque as that of a disk (τ=32ηωR³/3, where η is the viscosity), one obtains ω=3Nνμₒχ″H²/32ηR³, and, with a microwheel radius R that is proportional to the square root of the number of beads N, the rotation rate w is thus proportional to the inverse of the radius R. Similarly, with P=τω, the power P is proportional to the product of the rotation rate w and the number of beads N, and thus proportional to the radius R. This linear dependence on size drives the need for larger microwheels that can perform more mechanical work, which is defined as the application of power per unit time. In addition to the available power, the microwheels move at a velocity V that is proportional to the product of the rotation rate ω and the number of beads N, leading to the velocity V being proportional to the radius R and thus larger microwheels translating faster, as illustrated by the upper data points and line of FIG. 1B). For these reasons, while analogous nanoscale robots could be inhaled, they are not capable of performing significant work or being easily driven to desired sites once delivered.

The methods and systems of the present disclosure overcome the issues associated with existing approaches, which use external fields to bias the impaction of inhaled nanoparticles, by enabling airborne transport of microbot building blocks. In embodiments of these methods and systems, microwheels are delivered by seeding droplets with individual beads having a particle size of about 4.5 μm for subsequent in situ assembly and travel down the airway of a subject. Assembly of microwheels in situ has significant advantages, as the building blocks are small enough to be aerosolized and delivered into the lung pathways of the subject. Additionally, slightly larger particles, e.g. particles having a size of more than about 6 μm, are generally less susceptible to macrophage scavenging, further motivating the use of larger microbots. A further advantage of the methods and systems of the present disclosure is that the assembly of the microbots is reversible—upon removal of the magnetic field, the microwheels disassemble into individual beads for elimination by the subject's natural mechanisms for eliminating dust and other foreign particles in the mucus lining.

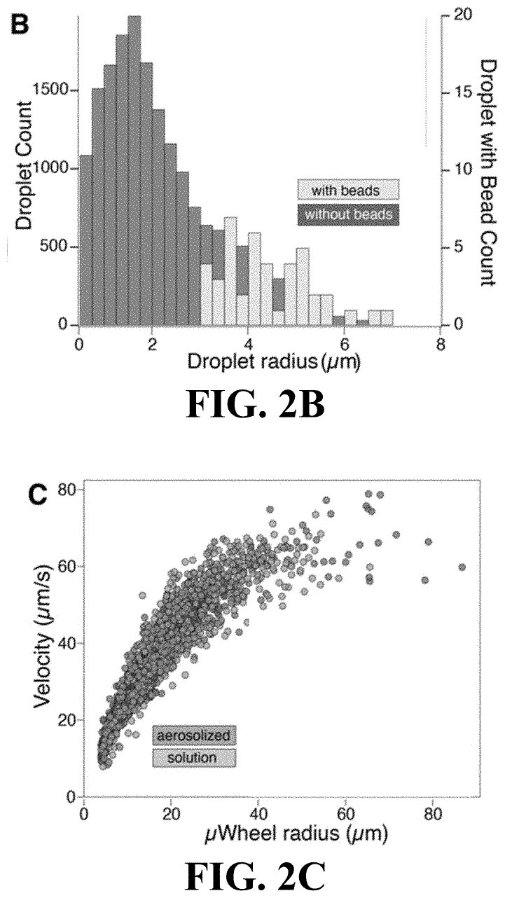

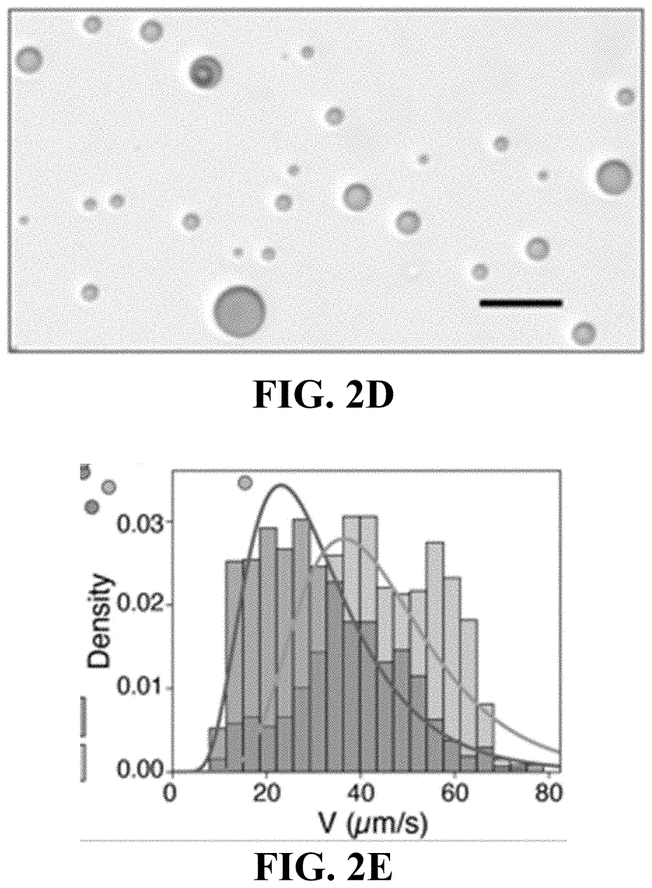

Referring now to FIG. 2A, one embodiment of a microbot delivery method according to the present disclosure utilizes aerosolized droplets that incorporate beads that, once delivered and upon application of a rotating magnetic field, assemble into microwheels. In this embodiment, beads randomly distribute within droplets having a size distribution controlled by a vaporizer; the liquid stream containing the beads is combined with a rapidly flowing air stream, and the ratio of the liquid stream to the air stream determines the droplet size distribution. This distribution is measured by directing aerosolized droplets into oil for subsequent imaging via optical microscopy, as illustrated in FIG. 2D, where both droplet size and particle containing distributions can be determined. The distributions can be readily varied by altering the relative air/liquid flow rates, the concentration of one or more surfactants, and/or the concentration of beads within the liquid stream. Once aerosolized, the droplets are directed to damp surfaces, where they impact, coalesce, and create a liquid film containing dispersed beads.

The primary mechanisms of aerosol delivery are inertial impaction, gravitational sedimentation, and Brownian diffusion. By these mechanisms, larger (e.g. more than about 5 μm) particles tend to embed in the upper airway, while smaller (e.g. less than about 0.1 μm) particles have the highest likelihood of being transported deep within the respiratory tract, for example the lower bronchioles and alveolar lung regions where particles would otherwise have to have a size of 10 nm or less to be transported via diffusion. While current aerosol-based drug delivery approaches utilize particle size in their targeting design, larger particles are required to do mechanical work or to translate effectively once embedded. While the present disclosure focuses mainly on embodiments in which the beads have a particle size in a range of between about 1 μm and about 5 the methods and systems disclosed herein overcome the drawbacks of deposition in the upper airway by enabling mechanisms to drive transport deeper into the lungs when desired.

Upon application of a weak rotating magnetic field, individual beads assemble into microwheels that move via wet friction. As illustrated in FIG. 2C, comparison of velocities of microwheels composed of beads from solution to velocities of microwheels assembled from aerosolized droplets shows that spraying does not negatively impact microwheel function. In the embodiment illustrated in FIG. 2C, droplets were initially formed within an aerosolizer and condensed on a surface in sufficient quantity to form a liquid layer. Within this layer and upon application of the magnetic field, the beads assembled into microwheels having a relationship between velocity and size (illustrated in FIG. 2C) similar to those assembled from solution. As illustrated in FIG. 2E, small differences in velocity distribution may arise due to local variation in bead concentration and resulting microwheel sizes during assembly.

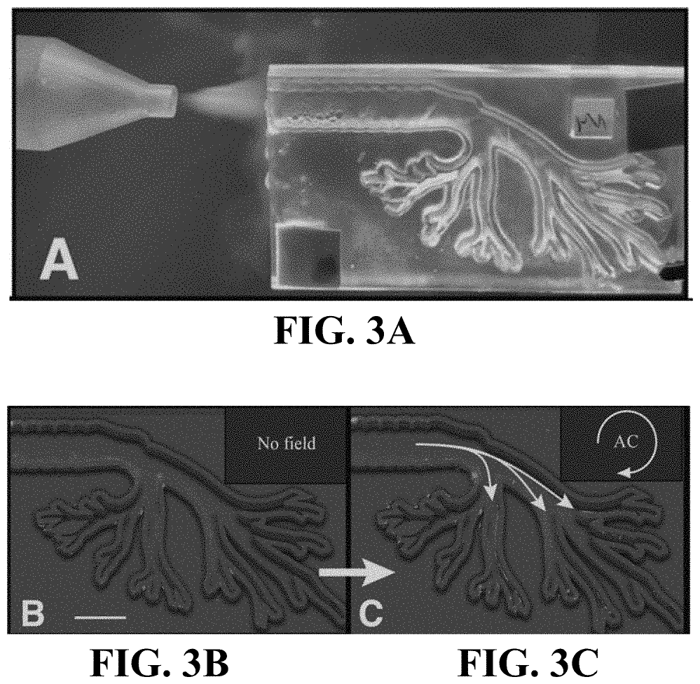

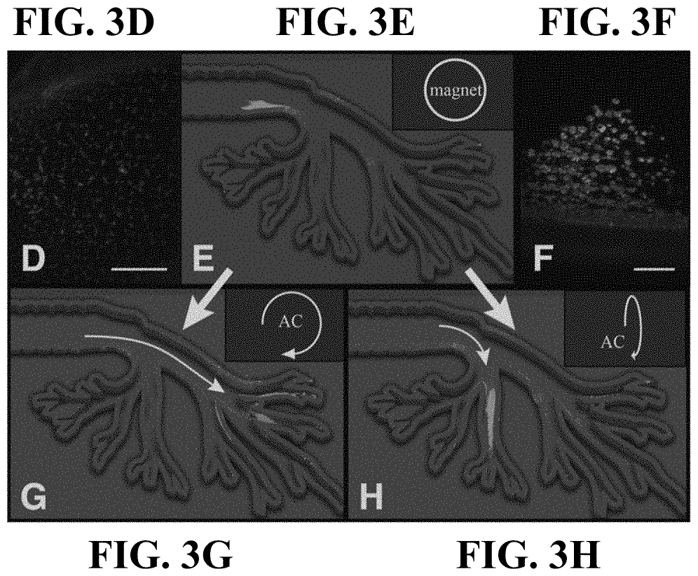

Referring now to FIG. 3A, aerosolized delivery of microbot building block beads within a 3D-printed human pediatric-scale lung mimic, fabricated at a length scale to model transport from the bronchiole into the alveoli, is illustrated. To aid imaging, the beads were fluorescently labeled and then aerosolized within droplets sprayed into the model using a commercially available nebulizer, dispersing broadly throughout the lung tissue model, as illustrated in FIG. 3B. Upon application of the rotating magnetic field, microwheels subsequently assembled and rolled down the bronchial tube to the lower bronchus within a time of 5 to 10 minutes, as illustrated in FIG. 3C. As illustrated in FIG. 3D, with the magnetic field directed to the right of the image, microwheels are driven to the end of the bronchial channels, where they accumulate.

While this transport capability is generally useful for delivering microwheels deeper into the lungs, more specific targeting may be useful for treatment of more localized diseases; for example, to avoid systemic delivery of chemotherapeutic agents and side effects associated therewith, inhaled delivery of drugs for the treatment of lung cancer could prove a promising approach. Previous progress toward successful implementation of these approaches has been limited due to concerns over toxicity and potential damage to healthy tissues throughout the rest of the lung, but an approach in which chemotherapeutic agents are delivered via microwheels to tumor surfaces could significantly enhance treatment and minimize side effects not only for the rest of the body but within the rest of the lung as well. To demonstrate this targeting ability, a bolus was created by placing a magnet within the end of the model inlet such that, upon aerosolization and entrance into the model lung, the beads aggregated, as illustrated in FIG. 3E. Upon removal of the magnet and application of the weak rotating magnetic field, microwheels formed, as illustrated in FIG. 3F, and could be directly driven to a desired endpoint within the model lung, as illustrated in FIGS. 3G and 3H. Because of the relatively large size of the aerosolized building blocks, using fixed magnets for targeting deep in the lungs is not likely to be a workable strategy for in vivo applications, but in practice such fixed magnets may not be necessary because the beads can naturally accumulate at the upper end of large-scale systems due to their size.

Interestingly, because of the high concentration of microwheels created by aerosolized delivery of the building blocks in the methods and systems of the present disclosure, swarming can be observed in the resulting assemblies. As has been shown elsewhere, such swarms can be actuated and controlled differently, enabling to net microbot transport optimized for dispersal, for travel up inclines, for speed, or for any of a wide variety of other transport characteristics. For the purposes of the measurements of transport in the embodiment illustrated in FIGS. 3A through 3H, the lung model was fixed horizontally, as gravity plays an important role in microbot transport, providing a load force and wet friction with adjacent surfaces. As may be expected, negative inclines increase translational velocities, while travel up steep slopes slows microbot movement; however, with appropriate field application, both individual microwheels and swarms of microwheels can continue to move up inclines with gradients as high as 80°.

Viscosity can also play a significant role in microwheel movement; given that the velocity V is proportional to the rotation rate ω for microwheels of a given size, it is to be expected that velocity V is thus also proportional to the inverse of the viscosity η and thus a slowing of the microwheels as viscosity increases. For travel from the bronchiole to the alveoli, a distance on the order of tens of centimeters, it is expected that microwheels will travel along the lower-viscosity sprayed fluid atop the higher-viscosity lung fluids, while transport distances through the thicker mucus layer are significantly shorter (typically up to no more than about a few hundred micrometers). The ability of these systems to deliver drugs and the incorporation of lung dispersants to lower local viscosities has already been demonstrated elsewhere.

Additionally, while the present disclosure focuses generally on the use of a nebulizer to aerosolize droplets, it is to be expressly understood that particles could be delivered in any of several other forms, for example as a dry powder, and such embodiments are within the scope of the present disclosure. Because the solid-phase building blocks are small enough and the particle size distribution sufficiently well defined, such approaches, once formulated, could provide certain advantages, such as eliminating the need for propellants or improving the effectiveness of delivery of certain classes of drug.

The present disclosure thus provides methods and systems for in situ microbot assembly that enable the delivery of microbots of substantial size and power into the airways of a subject's lung. The disclosure shows the feasibility of aerosolizing microbot building blocks by partitioning individual colloidal beads into droplets that are small enough to be delivered deep within pulmonary passages. With application of a weak rotating magnetic field, these individual particles assemble into larger microwheels capable of rapid translation through a model pulmonary network.

The inventive aspects of the present disclosure are further described and illustrated by way of the following non-limiting Examples.

### Example 1

### Magnetic Fields and Translation Studies

To create and control an applied rotating magnetic field, a magnetic field actuation system was constructed using coils and signal generation software. The z-axis of the actuation system consisted of one 400-turn coil (inner diameter 50 mm) positioned below the sample, and the x- and y-axes each consisted of two 400-turn coils (inner diameter 50 mm) positioned on either side of the sample. The field strength and frequency were kept constant at 4.7 mT and 40 Hz, respectively. For translation studies, the sample chamber consisted of two 22 mm×22 mm glass cover slips, each having a thickness of 0.17 mm, sandwiched with a 0.5 cm (inner diameter) gasket cut from double-sided tape (3M RP32 VHB™ tape). To this, a 12 μL sample of 4.5 μm diameter superparamagnetic beads (Thermo Fisher Dynabeads® M-450 epoxy, density 1.5 g/cm³) suspended in 0.2% sodium dodecyl sulfate (SDS) solution was added. The remainder of the gasket volume was then filled with 0.2% SDS solution before sealing.

FIG. 1A illustrates microwheel rotation and translation over a period of 2 seconds with the applied AC magnetic field at a magnetic flux density 3.37 mT; the scale bar represents 50 μm. It is observed that larger microwheels translate faster than smaller ones. FIG. 1B illustrates velocity (upper data points and line) and power (lower data points and line) dependence on assembled microwheel size, with linear fits to expected behaviors. FIG. 1C illustrates that the microwheel rotation rate co scales with the inverse of microwheel radius R.

### Example 2

### Bead-Laden Droplet Characterization

100 μL of 4.5 μm epoxy Dynabeads® at an initial concentration of about 4·10⁸ beads per mL were fluorescently labeled by addition to 200 μL of aqueous 1 mg/mL rhodamine B solution and 700 μL of 0.2 wt % SDS aqueous solution. After 24 hours at room temperature, this solution was washed with 0.2 wt % SDS solution a total of six times. 100 μL of this solution was then washed three times with 0.1 wt % SDS and 5 vol % glycerin. The final solution was made after discarding the supernatant and adding 500 μL of 0.05 wt % SDS, 5 vol % glycerin, and 50 mg/mL of green food dye to increase the contrast and to form spherical droplets without air inclusions. The aerosol was created using a Pari LC® Sprint Reusable Nebulizer (3.5 μm mass median diameter (MMD)) with supply air at 3 L/min. For quantification of droplet size, the aerosol was sprayed over a thin layer of Type B immersion oil on a glass slide for 1 minute. A brightfield macroscan of about 2 mm² was taken with a 20× objective. This scan was performed using a stage loop, where the camera and light source raster across a large area before being stitched together in software. Using threshold image analysis, the location and size of droplets and beads were determined. The data were then processed using a custom MATLAB script to assign each bead to a specific droplet; droplets below 0.5 μm in radius were not recorded due to image resolution limits.

FIG. 2A is a concept illustration of the microwheel assembly strategy. FIG. 2B is a graph of the measured size distribution of the aerosolized droplets, with bead-containing droplets identified; the overall fraction of droplets containing beads was 0.235%. FIG. 2D is an optical microscopic image (the scale bar represents 20 μm) of a sample of the aerosol in oil.

### Example 3

### Aerosolized Microwheel Velocities

4.5 μm epoxy Dynabeads® were diluted to a concentration of 4·10⁶ beads per mL with 0.2% SDS. The Pari LC® Sprint Reusable Nebulizer was positioned 2 cm away from, and disposed at a 45° angle relative to, a 22 mm×22 mm glass cover slip surrounded by a 5 mm-high 3D-printed retaining wall. The nebulizer was operated at 3.5 L/min air supply until about 1 mL of solution was collected on the cover slip. The beads were then assembled into microwheels and recorded using the magnetic actuation system described in Example 1 and an Olympus OpenStand microscope; the circular rotating magnetic field strength and frequency were 2.10 mT and 40 Hz, respectively. As a control, 1 mL of the same solution was pipetted onto an identical cover slip with retaining wall, then actuated under the same field conditions. The microwheel velocities were measured using available open-source tracking software, excluding stuck beads and monomers (defined as having a velocity of less than 5 μm/s).

FIG. 2C is a graph of pre- and post-aerosolization microwheel sizes and velocities; it is observed that both pre- and post-aerosolization microwheels demonstrate similar behavior with size. FIG. 2E is a histogram of velocities showing that the microwheel distribution post-aerosolization is shifted to smaller sizes.

### Example 4

### 3D Printing Lung Model and Targeting

The 3D-printed model lung was designed with a tracheal diameter of about 8 mm, corresponding to measured tracheal diameters in human infants. The clear model was 3D-printed (Form 3, FormLabs) and consisted of two halves that could be separated for cleaning and viewing. A new model was printed for each experiment to avoid residual fluorescence staining. The model was first prepared by wetting with about 2 mL of 0.2% SDS solution, then fluorescently labeled Dynabeads® were diluted to a concentration of 4·10⁷ beads/mL with 0.2% SDS solution before aerosolizing. The nebulizer nozzle was placed at the entrance of the model during aerosolization for 5 minutes. For experiments demonstrating targeting, a small permanent magnet was placed at the bottom of the model trachea.

For imaging, a fluorescence microscope with a motorized stage (Olympus IX81) was used to perform a full macroscan using a TRITC filter to characterize the initial distribution of beads throughout the entire model. For actuation, the device was placed on a separate fluorescence microscope (Olympus OpenStand) outfitted with magnetic actuation equipment; the magnetic field conditions were the same as those described in Example 3. Beads were assembled and microwheels actuated for 10 minutes. The rolling direction of the microwheels was changed manually according to the targeted bronchial branch. Finally, a second full macroscan of the device was performed to observe the movement of the fluorescently labeled beads after actuation.

FIG. 3A is an illustration of aerosolization of the bead-containing droplets into the 3D-printed lung model. FIG. 3B is a false-color image, with an illustrative overlay, of fluorescent, superparamagnetic beads dispersed throughout the model lung after aerosolization (the scale bar represents 1 cm). FIG. 3C is an illustration of the lung model showing that, upon application of the rotating magnetic field, microwheels form. FIG. 3D is an illustration of the microwheels traveling down the pathways of the model lung (the scale bar represents 1 mm). FIG. 3E is an illustration of the lung model showing that, for targeting, a permanent magnet can be used to capture aerosolized beads to form a bolus. FIG. 3F is an illustration of the model lung showing that, upon magnet removal and with subsequent application of a weak AC magnetic field, bolus microwheels can be driven to desired branches (the scale bar represents 1 mm). FIGS. 3G and 3H are illustrations of the model lung showing successful targeting of the bolus microwheels to specific branches or pathways within the model lung.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1. A method for delivering microrobots, comprising:
(a) providing a liquid suspension comprising superparamagnetic particles of at least one microrobot building block;
(b) aerosolizing the liquid suspension to form aerosolized droplets;
(c) administering the aerosolized droplets into a target volume; and
(d) applying a first magnetic field to the aerosolized droplets to cause the superparamagnetic particles to aggregate into microrobots in the form of microwheels.

2. The method of claim 1, wherein the target volume is within a body of an animal subject and the microrobots are medical microrobots capable of performing a medical intervention within the target volume.

3. The method of claim 2, wherein the animal subject is a human.

4. The method of claim 2, wherein the target volume comprises a pathway or space within a pulmonary or respiratory system of the animal subject.

5. The method of claim 4, wherein the target volume comprises at least one of lower bronchioles of the animal subject and alveolar spaces of the animal subject.

6. The method of claim 2, wherein the medical intervention comprises delivering an active pharmaceutical ingredient to the target volume.

7. The method of claim 1, further comprising:
directing the microwheels to a preselected sub-volume of the target volume by applying a second magnetic field.

8. The method of claim 1, wherein the liquid suspension is aerosolized, and the aerosolized droplets are administered, using a nebulizer.

9. The method of claim 1, wherein a mean or median particle size of the superparamagnetic particles is between about 1 μm and about 5 μm.

10. A method for performing a medical intervention within a body of an animal subject, comprising:
(a) providing a liquid suspension comprising superparamagnetic particles of at least one microrobot building block;
(b) aerosolizing the liquid suspension to form aerosolized droplets;
(c) administering the aerosolized droplets into a target volume within the body of the subject; and
(d) applying a first magnetic field to the aerosolized droplets to cause the superparamagnetic particles within the target volume to aggregate into microrobots in the form of microwheels,
wherein the microrobots are medical microrobots capable of performing a medical intervention within the target volume.

11. The method of claim 10, wherein the animal subject is a human.

12. The method of claim 10, wherein the target volume comprises a pathway or space within a pulmonary or respiratory system of the animal subject.

13. The method of claim 12, wherein the target volume comprises at least one of lower bronchioles of the animal subject and alveolar spaces of the animal subject.

14. The method of claim 10, wherein the medical intervention comprises delivering an active pharmaceutical ingredient to the target volume.

15. The method of claim 10, further comprising:
directing the microwheels to a preselected sub-volume of the target volume by applying a second magnetic field.

16. The method of claim 10, wherein the liquid suspension is aerosolized, and the aerosolized droplets are administered, using a nebulizer.

17. The method of claim 10, wherein a mean or median particle size of the superparamagnetic particles is between about 1 μm and about 5 μm.

18. A system for delivering microrobots, comprising:
a liquid suspension comprising superparamagnetic particles of at least one microrobot building block;
a nebulizer, configured to aerosolize the liquid suspension to form aerosolized droplets and administer the aerosolized droplets into a target volume; and
a magnetic actuation device, configured to apply a first magnetic field to the aerosolized droplets to cause the superparamagnetic particles within the target volume to aggregate into microrobots in the form of microwheels.

19. The system of claim 18, wherein the target volume is within a body of an animal subject and the microrobots are medical microrobots capable of performing a medical intervention within the target volume.

20. The system of claim 19, wherein the medical intervention comprises delivering an active pharmaceutical ingredient to the target volume.

21. The system of claim 18, wherein the magnetic actuation device is further configured to direct the microwheels to a preselected sub-volume of the target volume by applying a second magnetic field.

22. The system of claim 18, wherein a mean or median particle size of the superparamagnetic particles is between about 1 μm and about 5 μm.
